(19) United States
(12) Patent Application Publication
(10) Pub. No.: US 2023/0372124 A1
(43) Pub. Date: Nov. 23, 2023

(54) MUSCLE-DRIVEN ENDOPROSTHESIS SYSTEMS AND METHODS FOR USING SAME

(71) Applicant: UNIVERSITY OF TENNESSEE RESEARCH FOUNDATION, Knoxville, TN (US)

(72) Inventors: Dustin Lee Crouch, Knoxville, TN (US); David Edgar Anderson, Knoxville, TN (US); Stacy Stephenson, Knoxvile, TN (US)

(73) Assignee: UNIVERSITY OF TENNESSEE RESEARCH FOUNDATION, Knoxville, TN (US)

(21) Appl. No.: 18/197,849

(22) Filed: May 16, 2023

Related U.S. Application Data
(60) Provisional application No. 63/364,894, filed on May 18, 2022.

(51) Int. Cl. A61F 2/48; A61B 17/72; A61F 2/08

(52) U.S. Cl. CPC A61F 2/48; A61B 17/72; A61F 2/08; A61F 2002/30624

(57) ABSTRACT

A muscle-driven endoprosthesis system may include a bone extension, an intramedullary stem extending form a first end of the bone extension, a joint at a second end of the bone extension, a moveable portion coupled to the hinge, and synthetic tendons coupled to the moveable portion. The bone extension, intramedullary stem, hinge, and movable portion are sized and shaped to fit within a skin of a patient.

### RELATED APPLICATIONS

This application claims the benefits of U.S. Provisional Patent Application No. 63/364,894, titled “COMPLETELY IMPLANTED, UNJOINTED FOOT-ANKLE ENDOPROSHTETIC LIMB IN RABBIT MODEL,” filed May 18, 2022, the disclosure of which are incorporated herein by reference in their entireties.

### STATEMENT REGARDING FEDERALLY SPONSORED RESEARCH

This invention was made with United States Government support under Grant No. R61AR078096 awarded by the National Institutes of health and Grant No. 1944001 awarded by the National Science Foundation. The United States Government has certain rights in the invention.

### BACKGROUND

Limb amputation severs the physical connection between muscles and limb segments, causing severe, extensive sensorimotor impairment for about 2 million Americans, a population that increases by nearly 185,000 annually. An externally worn limb prosthesis is the standard means to restore part of the missing limb's function. Unfortunately, according to recent surveys of amputees, limb prostheses still do not move or feel like the biological intact limb. Consequently, as many as 45% and 35% of users of body-powered and motorized prostheses, respectively, eventually abandon their prosthesis.

The physiologic sensorimotor pathway is a complex neural network that enables the perception and control of movements. It involves the interaction between sensory inputs and motor outputs and is responsible for the coordination of sensory information and motor responses. A component of the physiologic sensorimotor pathway is the physical link between muscles and limb segments, which is severed by amputation. When stimulated by efferent motor commands from the nervous system, muscles contract and transmit force across one or more joints to move limb segments. When subsequently stretched or loaded, muscles contribute to proprioception and reflexes through afferent feedback from mechanoreceptors, such as muscle spindles and Golgi tendon organs. Because the muscles are physically connected to the moving limb segments, there is close coupling, or alignment, among efferent motor commands (representing movement intent), the actual movement, and the sensed movement via afferent proprioceptive feedback. Such sensorimotor alignment provides for effective closed-loop motor control in normal muscle movements. Limb amputation breaks the physical link between muscles the missing limb segments, severely disrupting sensorimotor alignment. This possibly explains why amputees' “phantom” sense of the missing limb's size, posture, and movement is distorted or absent.

Current prosthetics and prosthetic systems are less than ideal for a number of reasons. For example, state-of-the-art electromechanical prostheses, which substitute for muscles in the sensorimotor pathway using electric motors and other hardware, cannot yet restore true sensorimotor alignment. This is because electromechanical prosthesis substitute for muscles at many points along the physiologic sensorimotor pathway. Because electromechanical systems do not yet perfectly account for or mimic physiologic processes, they inevitably interject some error (with respect to the user's movement intent) at each substituted point on the sensorimotor pathway. For example, motor commands can vary across limb postures for a given movement. Estimates of movement intent or proprioceptive stimulation cannot be rigorously validated and, thus, likely conflict with the user's actual intent. Additionally, synthetic (e.g., electric) motors cannot yet mimic the force-generating behavior of muscles. Any errors lead to sensorimotor misalignment, or inconsistency among the intended, actual, and sensed movements, which limits users' function and satisfaction with current prosthetics and prosthetic systems.

The concept of physically attaching muscles to prostheses has been investigated in the past. However, identifying a practical and widely accepted approach remains an open challenge. Since all existing limb prostheses are worn external to the body, physical muscle-prosthesis attachment requires the muscle force to be externalized in some way. This was initially achieved using cineplasty, a surgically created skin-tendon loop that can be attached to an external prosthesis by a cable. Cineplasty purportedly enabled control performance near that of the intact limb.

However, cineplasty has limitations in function and appearance that have deterred widespread clinical use. In terms of function, part of the muscle force is diverted toward stretching the skin itself rather than moving the prosthesis. Additionally, cineplasty may not lengthen muscles to their pre-amputation lengths, limiting excursion (i.e., range of lengths over which a muscle can produce active force) and maximal force (i.e., muscle mass).

In light of the above, improved devices and methods that overcome at least some of the above limitations of the prior devices and methods would be helpful.

### SUMMARY

Embodiments of the present disclosure provide improved systems and methods including the use of muscle-driven, fully implanted, endoprosthesis that enables closed-loop physiologic sensorimotor feedback and provide for improved outcomes for amputation patients.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows an intact and amputated extremity, in accordance with some embodiments herein;

FIG. 2 shows external prosthesis, in accordance with some embodiments herein;

FIG. 3 shows a muscle-driven ankle endoprosthesis, in accordance with some embodiments herein;

FIG. 4 shows a muscle-driven finger endoprosthesis, in accordance with some embodiments herein;

FIG. 5 shows a muscle-driven wrist endoprosthesis, in accordance with some embodiments herein;

FIG. 6 shows various muscle-driven endoprosthesis, in accordance with some embodiments herein;

FIG. 7 shows a method of using a various muscle-driven endoprosthesis, in accordance with some embodiments herein;

FIG. 8 an experimental muscle-driven endoprosthesis, in accordance with some embodiments herein; and

FIG. 9 an experimental muscle-driven endoprosthesis, in accordance with some embodiments herein.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

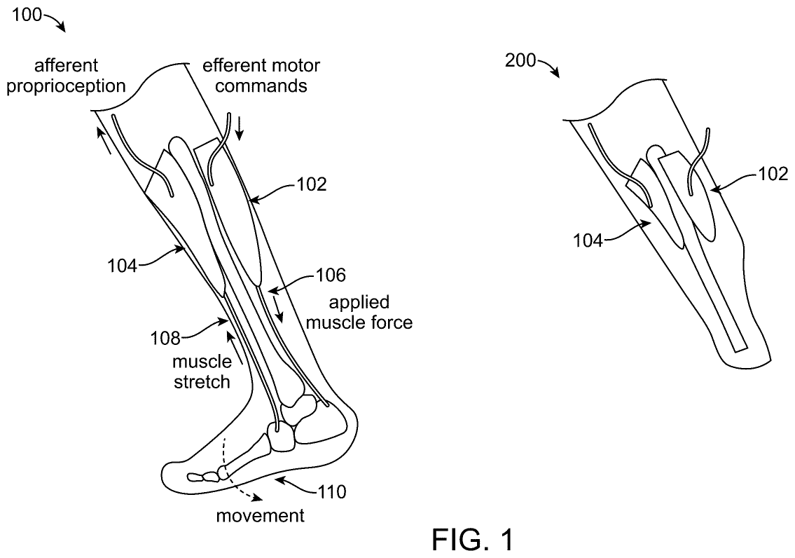

FIG. 1 shows an intact extremity 100 and an amputated extremity 200. Although a lower leg, ankle, and foot are shown and described herein, the discussion herein may apply to other extremities. Each extremity includes bones, joints, muscles, nerves, and other tissues that work together to allow movement and provide support for the body. They also play an important role in sensation, helping to feel and interact with the environment through touch and proprioception. Other extremities may include the arms, hands, legs, and feet. The arms are the upper extremities of the human body and include the upper arm, forearm, and hand. The upper arm extends from the shoulder to the elbow and contains the humerus bone, while the forearm extends from the elbow to the wrist and contains the radius and ulna bones. The hand includes the wrist, palm, fingers, and thumb. The upper extremities are used for grasping, manipulating objects, and balance, among other tasks.

The legs are the lower extremities of the human body and include the thigh, lower leg, and foot. The thigh extends from the hip joint to the knee joint and includes the femur bone, while the lower leg extends from the knee to the ankle joint and include the tibia and fibula bones. The foot includes the ankle, heel, sole, toes, and arch. The lower extremities are used for standing, walking, running, among other tasks.

A component of the physiologic sensorimotor pathway is the physical link between muscles and limb segments. When stimulated by efferent motor commands from the nervous system, muscles contract and transmit force across one or more joints to move limb segments. They are an aspect of the motor system and are responsible for generating and executing movement. Efferent motor commands are signals that originate in the central nervous system (CNS) and are transmitted to the peripheral nervous system (PNS) to control the activity of muscles and other effectors. Efferent motor commands are generated in the motor cortex of the brain and are transmitted through descending pathways in the spinal cord to the motor neurons in the PNS. These motor neurons then transmit the commands to the muscles, where they initiate and modulate muscle contractions to produce movement.

When muscles are subsequently stretched or loaded in response to motor commands, muscles contribute to proprioception and reflexes through afferent feedback from mechanoreceptors, such as muscle spindles and Golgi tendon organs. Afferent feedback from mechanoreceptors includes sensory information that is transmitted from specialized sensory receptors in the body to the central nervous system to provide information about the position, movement, and force of body parts. These sensory receptors, known as mechanoreceptors, are located in muscles, joints, tendons, and skin, and are responsible for detecting mechanical stimuli such as pressure, stretch, and vibration.

When these mechanoreceptors are activated, they generate electrical signals that travel through afferent nerve fibers to the spinal cord and brainstem, where they are processed and integrated to produce a perceptual representation of the body's position and movement. This information is then used to adjust the efferent motor commands that control movement, enabling precise and coordinated movement.

Because the muscles are physically connected to the moving limb segments, there is close coupling, or alignment, among efferent motor commands (representing movement intent), the actual movement, and the sensed movement via afferent proprioceptive feedback. Such sensorimotor alignment plays a large role in effective closed-loop motor control. Disruption of sensorimotor alignment can severely impair performance of motor tasks, especially in the upper extremity.

In the intact limb 100, for example, efferent motor commands from the nervous system cause the calf muscles 102, such as the gastrocnemius and soleus muscles to contract. The gastrocnemius and soleus muscles are two muscles in the posterior compartment of the leg that play a role in the movement and stability of the ankle and foot. The gastrocnemius muscle is a large, superficial muscle that originates from the femur bone above the knee joint and attaches to the calcaneus bone in the foot via the Achilles tendon 106. It is primarily responsible for plantar extension of the ankle, which is the movement that points the toes downward, and for providing stability to the ankle joint during weight-bearing activities such as standing and walking.

The soleus muscle is a deep muscle that lies underneath the gastrocnemius muscle and also originates from the tibia and fibula bones in the lower leg. It attaches to the calcaneus bone via the Achilles tendon 102, just like the gastrocnemius muscle. The soleus muscle is also involved in plantar extension of the ankle, but it is more active during activities that require endurance, such as running and cycling.

The muscles contract in response to the efferent motor commands and transmit force across one or more joints, such as the ankle joint to move limb segments, such as the foot 110.

Because the limb is intact, the muscles are physically connected to the moving limb segments and there is close coupling, or alignment, among efferent motor commands representing movement intent, the actual movement, and the sensed movement via afferent proprioceptive feedback.

When the calf muscles 102 contract, the foot is extended, causing the tibialis anterior muscle 104 to stretch. The tibialis anterior originates from the upper two-thirds of the tibia bone and the interosseous membrane (a thin sheet of connective tissue between the tibia and fibula) and inserts into the medial cuneiform bone and the base of the first metatarsal bone of the foot. The tibialis anterior muscle is responsible for dorsiflexion of the ankle joint, which is the movement that lifts the foot upward towards the shin.

The afferent proprioceptive feedback resulting from the stretching of the tendon 108 and tibialis anterior muscle 104 is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

Limb amputation breaks the physical link between muscles the missing limb segments, severely disrupting sensorimotor alignment. This in part may explain why amputees' “phantom” sense of the missing limb's size, posture, and movement is distorted or absent. The severing also significantly impairs a person's ability to sense the position of and use external prostheses.

Amputated limb 200 shows the lack of connection between the missing extremity and the shin muscles 104 and calf muscles 102.

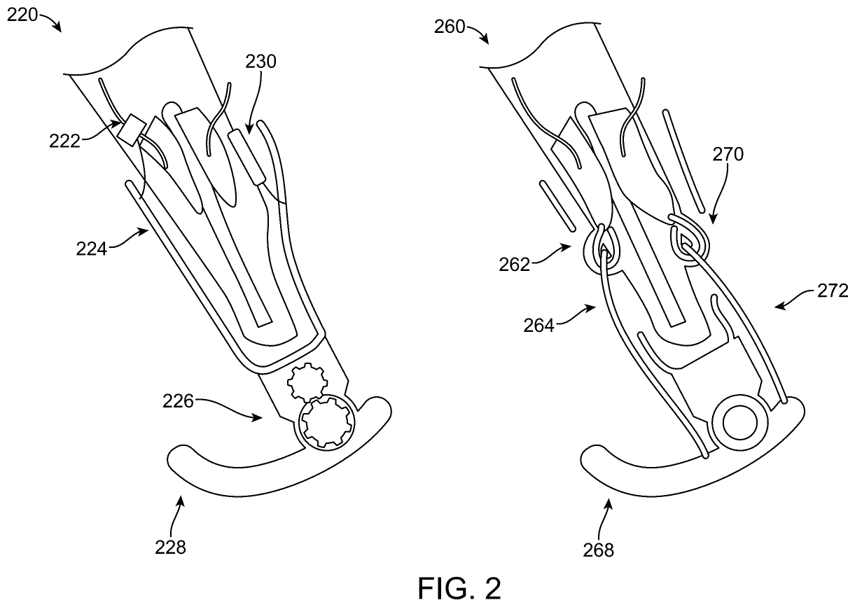

FIG. 2 shows a motor-driven external prostheses 220 and a cineplasty external prosthesis 260. In the motor-driven prostheses example, the external prostheses includes a prosthetic extremity 228 coupled to a motorized joint 226. The external prosthesis may then be externally attached to a limb either by a socket that fits over the residual limb, or to a bone-anchored stem that protrudes out of the skin. The external electromechanical prosthesis 220 substitutes for muscles at many points along the physiologic sensorimotor pathway. For example, an EMG measurement device 230 may be implanted or attached to the patient's tissues to measure motor commands (e.g., electromyograms). An EMG measurement device 230, also known as an electromyography device, is a medical instrument that is used to measure and record the electrical activity of muscles. The device may include a set of electrodes that are placed on the skin or inserted into the muscle tissue, and a recording unit that amplifies and records the electrical signals generated by the muscles.

The electrical signals generated by muscles are called electromyograms, or EMGs, and are produced when the muscle fibers contract. By measuring the EMGs, the device can provide information about the activity, strength, and timing of muscle contractions, as well as the patterns of muscle activation during movement. This information may then be used to estimate the user's movement intent from motor commands, convert movement intent estimates into control outputs, and generate movements with electric motors based on the control outputs. Sensory stimulation may be provided by a sensory stimulator 222 that may evoke proprioceptive feedback by applying electrical stimulation through electrodes to peripheral or cortical sensory neurons or by mechanical stimulation to the skin. Because electromechanical systems do not yet perfectly account for or mimic physiologic processes, they inevitably interject some error (with respect to the user's movement intent) at each substituted point on the sensorimotor pathway. For example, motor commands can vary across limb postures for a given movement. Estimates of movement intent or proprioceptive stimulation cannot be rigorously validated and, thus, likely conflict with the user's actual intent. Additionally, synthetic (e.g., electric) motors cannot yet mimic the force-generating behavior of muscles. Errors in sensing intent or providing stimulation lead to sensorimotor misalignment, or inconsistency among the intended, actual, and sensed movements, which limits users' function and satisfaction with a prosthesis.

The cineplasty external prostheses 260 is attached to the muscles through skin-tendon loops 262, 270 using Cineplasty. Cineplasty involves surgically creating skin-tendon loop 262, 270 that may be attached to an external prosthesis 260 by a cable 264, 272. Cineplasty reportedly enabled control performance near that of the intact limb using a physical muscle-prosthesis link that restored sensorimotor alignment. However, cineplasty has limitations in function and appearance that have deterred widespread clinical use. In terms of function, part of the muscle force is diverted toward stretching the skin itself rather than moving the prosthesis. Additionally, cineplasty may not lengthen muscles to their pre-amputation lengths, limiting excursion (i.e., range of lengths over which a muscle can produce active force) and maximal force (i.e., muscle mass). Though sensors and motors could amplify weak residual muscle output, it would be mechanically simpler and less costly to implement if the residual muscles had enough inherent force-generating capacity to enable useful motor function.

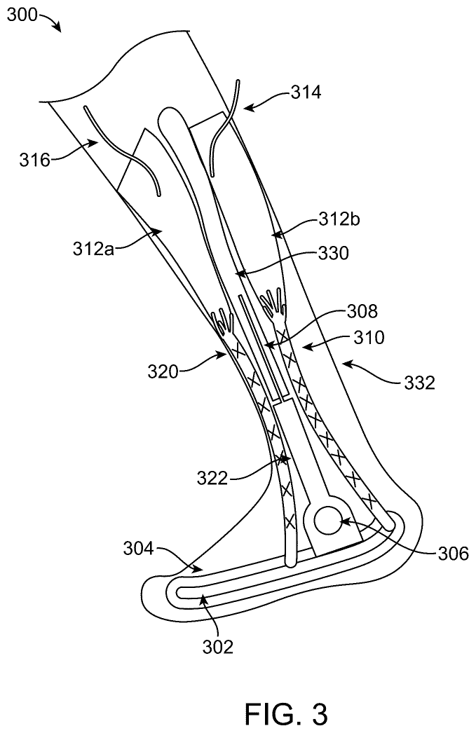

FIG. 3 shows an internal muscle-driven ankle endoprosthesis 300. The endoprosthesis 300 may include an intramedullary stem 308 to anchor the endoprosthesis 300 in the distal end of the tibia bone. An intramedullary stem 308 may be used as an anchoring device for the prostheses. The intramedullary stem may also be referred to as a prosthetic stem, bone stem, osseointegration implant, or osseointegrated implant.

Over time, the intramedullary stem fuses with the surrounding bone tissue, providing a strong and stable anchoring point for the rest of the endoprosthesis 300. The intramedullary stem is inserted into a hole drilled into the bone, and over time, the bone tissue grows and fuses with the surface of the implant, creating a strong and stable bond.

The intramedullary stem may be made from a biocompatible material, such as titanium or a titanium alloy. Other materials that may be used include ceramics, such as hydroxyapatite, and synthetic polymers. These materials may have different properties than metals, such as a lower modulus of elasticity or greater porosity and may be used in certain situations where different properties are desired.

The structure of the intramedullary stem can also vary depending on the application. The intramedullary stem may be screw-shaped with threads designed to be screwed into bone tissue. In some embodiments, the intramedullary stem may be cylindrical or have other shapes. The intramedullary stem 308 may have a roughened surface. Roughened surfaces have a texture or topography that promotes bone growth and attachment. The surface roughness may be achieved by various methods, such as sandblasting, acid etching, or plasma spraying.

The surface of the intramedullary stem may be porous. Porous implants have a porous structure that allows bone tissue to grow into the implant and anchor it in place. These implants can be made using various methods, such as powder metallurgy or additive manufacturing techniques like 3D printing.

The surface of the implant may also be treated with coatings or other treatments to enhance osseointegration and improve the stability of the implant. Hydroxyapatite is a mineral that is naturally found in bone tissue. Hydroxyapatite coatings may be applied to the surface of implants to enhance their biocompatibility and promote osseointegration. The hydroxyapatite coating provides a surface that mimics the natural mineral composition of bone tissue, which encourages bone growth and attachment.

The intramedullary stem may have a bioactive glass coating. Bioactive glass is a type of glass that is designed to interact with biological tissues. The glass can be coated onto the surface of implants to provide a surface that promotes osseointegration. The bioactive glass coating can also release ions into the surrounding tissue, which can further enhance the bone growth process.

The intramedullary stem may have a titanium oxide coating. Titanium oxide is a naturally occurring material that can be applied as a coating to titanium implants. The coating provides a surface that promotes osseointegration and improves the biocompatibility of the implant.

The intramedullary stem may have a zinc coating. Zinc is a trace element that aids in the natural bone growth and repair processes. Zinc coatings can be applied to the surface of implants to promote bone growth and attachment. Zinc coatings can also provide antimicrobial properties, which can help to reduce the risk of infection.

The intramedullary stem may be coupled to a prosthetic bone extension 322. The prosthetic bone extension 322 may extend from the end of a natural bone to a joint 306 of the endoprosthesis 300. The prosthetic bone extension 322 may add length of the extremity such that it matches the length of the original extremity. In some embodiments, the length of the bone extension may be chosen such that, in combination with the rest of the endoprosthesis, the limb or extremity is the same length as the original, non-amputated limb or extremity of the surviving symmetrical limb or extremity. For example, if a right limb is amputated, then the bone extension may be of length such that the right limb matches the length of the unamputated or intact left limb.

The joint 306 may couple a limb portion of the prosthetic, such as the prosthetic bone extension, to an extremity prosthetic, such as the prosthetic foot portion 302. The joint 306 may be a 1-degree-of-freedom hinge joint that permits ankle dorsi and plantar flexion. In some embodiments, the joint may be a two degree of freedom joint that allows supination and pronation. The angular movement of the joint in one or both degrees of freedom may be limited. The range of motion for supination may be limited to less than 10 degrees, less than 15 degrees, less than 20 degrees, or less than 30 degrees in order to support the patient's body and prevent over supination. In some embodiments, the range of motion for pronation may be limited to less than 5 degrees, less than 10 degrees, or less than 15 degrees. The range of motion for dorsiflexion may be limited to less than 5 degrees, less than 10 degrees, less than 15 degrees, or less than 20 degrees. The range of motion for plantarflexion may be limited to less than 15 degrees, less than 20 degrees, less than 25 degrees, less than 30 degrees less than 35 degrees, less than 40 degrees, less than 45 degrees, or less than 50 degrees.

The range of motion for supination may exceed 10 degrees, 15 degrees, 20 degrees, or 30 degrees in order to support the patient's body. In some embodiments, the range of motion for pronation may exceed 5 degrees, 10 degrees, or 15 degrees. The range of motion for dorsiflexion may exceed 5 degrees, 10 degrees, 15 degrees, or 20 degrees. The range of motion for plantarflexion exceed 15 degrees, 20 degrees, 25 degrees, 30 degrees, 35 degrees, 40 degrees, 45 degrees, or 50 degrees.

The internal muscle-driven ankle endoprosthesis 300 may include a movable portion 302 that acts as the moveable extremity, such as the foot. The foot portion may be made from a biocompatible metal, such as titanium and its alloys. Titanium and its alloys may be used for their biocompatibility, high strength-to-weight ratio, and corrosion resistance. Titanium implants may be well-tolerated by the body and have a low risk of causing an immune response or rejection. The foot portion may be made from stainless steel. Stainless steel is strong, durable, and corrosion-resistant, and is biocompatible. The foot portion may be made from cobalt-chromium alloys. Cobalt-chromium alloys are strong and durable and have good resistance to wear and corrosion.

The foot segment may be shorter than the biological foot so that it fits within the native skin envelope surrounding the biological foot. The front end of the foot may be curved upward to permit biomechanical “roll-over” that occurs at the toes during locomotion.

The internal muscle-driven ankle endoprosthesis 300 may include a sleeve 304 that is over-molded onto the rigid segments, such as the foot portion, the joint, and the bone extension to provide a soft surface for the overlying skin. The sleeve provides a compliant skin-internal muscle-driven ankle endoprosthesis 300 interface that aids in reducing the risk of skin injury, the rigid segments may be over-molded with a liquid silicone rubber. In some embodiments, the sleeve may be a durable, non-degradable, biocompatible, soft, moldable material, such as silicone, PEEK, nylon, polypropylene, etc.). The sleeve may have a geometry as appropriate for the specific device design, implant location, etc. For example, the sleeve may have a thickness of between 1 mm and 12 mm thick, between 1 mm and 8 mm, preferably between 2 mm and 6 mm. In some embodiments, a thumb may have a sleeve thickness of between 2 mm and 5 mm.

The internal muscle-driven ankle endoprosthesis 300 may be coated with other types of biocompatible soft materials such as polyurethane, hydrogels, collagen elastomers, or other materials. Polyurethane is a highly versatile material that can be tailored with specific properties, such as strength, flexibility, and permeability. Hydrogels are soft, water-containing polymers. Hydrogels can mimic the natural environment of cells and tissues, making them highly biocompatible and suitable for use in the body. Collagen is a protein that is found in the connective tissues of the body, such as skin, bone, and cartilage. It can be processed into various forms, such as gels, films, and sponges. Elastomers are rubber-like materials are highly flexible and can withstand repeated stretching and compression without losing their shape.

The endoprosthesis 300 may include synthetic tendons 310, 320. The synthetic tendons may extend from a natural tendon or the muscles 312a, 321b to the moving portion of the endoprosthesis limb segment. The synthetic tendons may be made of biocompatible synthetic materials, such as polyester, polyethylene, or polyurethane, which are designed to mimic the strength and flexibility of natural tendons. The synthetic tendons may include multiple individual strands of the synthetic tendon. The synthetic tendon may include a plurality of braided suture strings, each braided suture string being a bundle of thin (12-μm diameter) polyester fibers. A first end of the synthetic tendons may be threaded into the distal ends of the muscles that contribute to ankle and plantar flexion in the biological ankle. A second end of the synthetic tendons may be coupled or secured to the movable portion 302 that acts as the moveable extremity, in this case the synthetic tendons are coupled to the prosthetic foot portion 302. A first of the synthetic tendons may be coupled to the foot portion 302 on a first side of the joint 306 proximate the heal portion of the foot portion 302 which may be a posterior portion of the foot portion 302. A second of the synthetic tendons may be coupled to the foot portion 302 on a second side of the joint 306, opposite the first side, such as a distal portion of the foot portion 302 which may be an anterior portion of the foot portion 302.

With the internal muscle-driven ankle endoprosthesis 300, the limb may act as an intact limb with respect to aspects of the efferent motor command and afferent proprioceptive feedback. For example, efferent motor commands from the nervous system cause the calf muscles 312b, such as the gastrocnemius and soleus muscles to contract. The contraction of the gastrocnemius and soleus muscles pull on the synthetic Achilles tendon 310, which causes plantar flexion of the ankle, rotating the foot 302 around the joint 306, causing the toe and ball end of the foot to move downward.

The soleus muscle is a deep muscle that lies underneath the gastrocnemius muscle and also originates from the tibia and fibula bones in the lower leg. It also attaches to the posterior portion of the foot via the synthetic Achilles tendon 310.

Because the limb has an internal muscle-driven ankle endoprosthesis 300, the muscles are physically connected to the moving limb segments and there is close coupling, or alignment, among efferent motor commands representing movement intent, the actual movement, and the sensed movement via afferent proprioceptive feedback.

When the calf muscles 312b contract, the foot is extended, causing the tibialis anterior muscle 312a to stretch. The tibialis anterior originates from the upper two-thirds of the tibia bone and the interosseous membrane (a thin sheet of connective tissue between the tibia and fibula) and is attached via synthetic tendon 320 to the anterior portion of the foot portion.

The afferent proprioceptive feedback resulting from the stretching of the tendon 312a is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

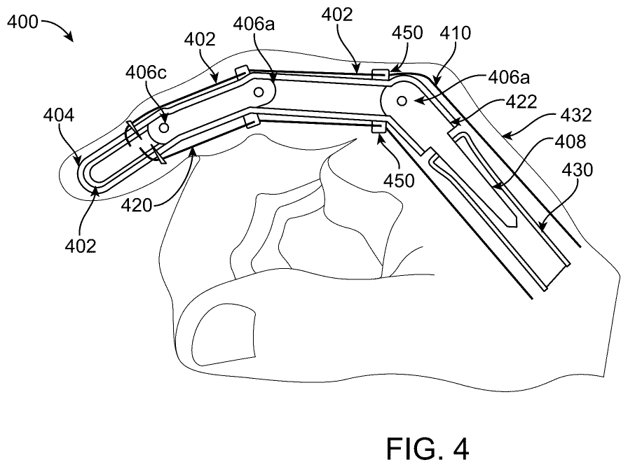

FIG. 4 shows an internal muscle-driven finger endoprosthesis 400. The endoprosthesis 400 may include an intramedullary stem 408 to anchor the endoprosthesis 400 in the distal end of a metacarpal bone 430. An intramedullary stem 408 may be used as an anchoring device for the prostheses. In some embodiments the intramedullary stem may be inserted into and anchored proximal phalange, middle phalange or the distal phalange. The intramedullary stem may also be referred to as an osseointegration implant or osseointegrated implant.

Over time, the intramedullary stem fuses with the surrounding bone tissue, providing a strong and stable anchoring point for the rest of the endoprosthesis 400. The intramedullary stem is inserted into a hole drilled into the bone, and over time, the bone tissue grows and fuses with the surface of the implant, creating a strong and stable bond.

The intramedullary stem may be made from a biocompatible material, such as titanium or a titanium alloy. Other materials that may be used include ceramics, such as hydroxyapatite, and synthetic polymers.

The structure of the intramedullary stem can also vary depending on the application. The intramedullary stem may be screw-shaped with threads designed to be screwed into bone tissue. In some embodiments, the intramedullary stem may be cylindrical or have other shapes. The intramedullary stem 408 may have a roughened surface. Roughened surfaces have a texture or topography that promotes bone growth and attachment. The surface roughness may be achieved by various methods, such as sandblasting, acid etching, or plasma spraying.

The surface of the intramedullary stem may be porous. The surface of the implant may also be treated with coatings or other treatments to enhance osseointegration and improve the stability of the implant. The intramedullary stem may have a bioactive glass coating. The intramedullary stem may have a titanium oxide coating. The intramedullary stem may have a zinc coating.

The intramedullary stem may be coupled to a prosthetic bone extension 422. The prosthetic bone extension 422 may extend from the end of a natural bone to a joint 406a of the endoprosthesis 400. The prosthetic bone extension 422 may add length of the extremity such that it matches the length of the original extremity. In some embodiments, the length of the bone extension may be chosen such that, in combination with the rest of the endoprosthesis, the finger or extremity is the same length as the original, non-amputated finger or extremity of the surviving symmetrical limb or extremity. For example, if a right finger is amputated, then the bone extension may be of length such that the right limb matches the length of the unamputated or intact left finger.

The joint 406a may be a first of multiple joints 406 that couple multiple movable prosthetic portions 402 together. The joints 406 may be a 1-degree-of-freedom hinge joint that permits finger flexion and extension. Finger flexion refers to the movement of bending the fingers towards the palm of the hand. The opposite movement, which involves straightening the fingers, is called finger extension or digital extension. In some embodiments, such as a prosthetic metacarpophalangeal joint, the joint may be a two degree of freedom joint that allows other types of finger movement. For example, abduction, the movement of spreading the fingers apart from each other, away from the midline of the hand, adduction the movement of bringing the fingers back together towards the midline of the hand, and circumduction, a combination of flexion, extension, abduction, and adduction, resulting in a circular motion of the finger around the metacarpophalangeal joint. The angular movement of the joint in one or more degrees of freedom may be limited. The range of motion may be limited to less than 15 degrees, less than 20 degrees, less than 25 degrees, less than 30 degrees less than 35 degrees, less than 40 degrees, less than 45 degrees, or less than 50 degrees. The range of motion exceed 15 degrees, 20 degrees, 25 degrees, 30 degrees, 35 degrees, 40 degrees, 45 degrees, or 50 degrees.

The internal muscle-driven finger endoprosthesis 400 may include one or more movable portions 402 that act as the moveable extremity, such as the finger. The finger portions may be made from a biocompatible metal, such as titanium and its alloys. The finger portions may be made from stainless steel. The finger portions may be made from cobalt-chromium alloys. These metals and alloys may be used for their biocompatibility, high strength-to-weight ratio, and corrosion resistance as well as because they are well-tolerated by the body and have a low risk of causing an immune response or rejection.

The finger portions may be shorter than the biological foot so that they fit within the native skin envelope surrounding the biological finger.

The internal muscle-driven finger endoprosthesis 400 may include a sleeve that is over-molded onto the rigid segments or moveable portions, such as the movable prosthetic portions 402, the joint, and the bone extension to provide a soft surface for the overlying skin. The sleeve provides a compliant skin-internal muscle-driven finger endoprosthesis 400 interface that aids in reducing the risk of skin injury, the rigid segments may be over-molded with a liquid silicone rubber.

The internal muscle-driven finger endoprosthesis 400 may be coated with other types of biocompatible soft materials such as polyurethane, hydrogels, collagen elastomers, or other materials.

The endoprosthesis 400 may include synthetic tendons 410, 420. The synthetic tendons may extend from a natural tendon or muscles to the moving portion of the endoprosthesis limb segment. The synthetic tendons may extend from one or more of the hypothenar, lumbricals, and abductor pollicis muscles of the hands and fingers. The synthetic tendons may be made of biocompatible synthetic materials, such as polyester, polyethylene, or polyurethane, which are designed to mimic the strength and flexibility of natural tendons. The synthetic tendons may include multiple individual strands of the synthetic tendon. A first end of the synthetic tendons may be threaded into the distal ends of the muscles that contribute to finger movement in the biological hand. A second end of the synthetic tendons may be coupled or secured to a movable portion 402 that acts as the moveable extremity, in this case the synthetic tendons are coupled to the prosthetic distal phalanx 402. A first of the synthetic tendons may be coupled to the prosthetic distal phalanx 402 on a first side such as a dorsal side. A second of the synthetic tendons may be coupled to the prosthetic distal phalanx 402 on a first side such as a palmar side.

In some embodiments, an endoprosthesis may have tendon guides 450. Tendon guides may be used between two joints or on a distal end of a distal most joint. Tendon guides may include an aperture through with the synthetic tendon passes. The tendon guide 450 may capture and retain the synthetic tendons and prevent or aid in preventing the synthetic tendons from slipping around the prosthetic. The tendon guides 540 may be used in situations when the tendon should curve around joint of a prosthetic or when the straight line distance between a first end of a synthetic tendon attached to a muscle and a second end of a tendon attached to a prosthetic is shorter than the path of the tendon along the prosthetic, such as when a finger bends. When a finger bends, the synthetic tendons should follow the path of the curved finger, but the shortest distance may be different. Without the tendon guides, the tendons may slip and cause discomfort or the prosthetic may fail to work as intended.

With the internal muscle-driven finger endoprosthesis 400, the finger may act as an intact finger with respect to aspects of the efferent motor command and afferent proprioceptive feedback. For example, efferent motor commands from the nervous system cause the finger muscles to contract. The contraction of the finger muscles pull on the synthetic tendon 420, which causes finger to bend.

Because the finger has an internal muscle-driven finger endoprosthesis 400, the muscles are physically connected to the moving finger and there is close coupling, or alignment, among efferent motor commands representing movement intent, the actual movement, and the sensed movement via afferent proprioceptive feedback.

When the finger muscles attached to synthetic tendon 420 contract, the finger bends, causing the synthetic tendon 410 to stretch the muscle or muscles to which it is attached to stretch.

The afferent proprioceptive feedback resulting from the stretching of the muscle or muscles to which the synthetic tendon 410 is attached is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

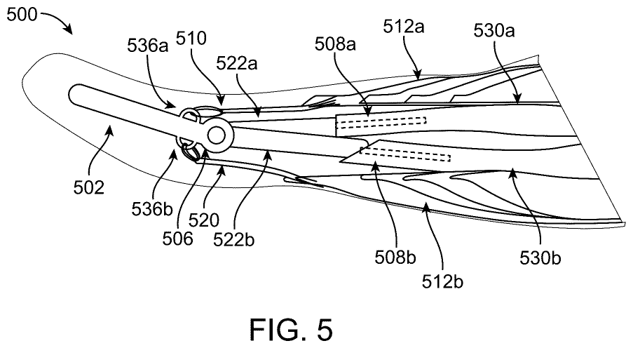

FIG. 5 shows an internal muscle-driven wrist and hand endoprosthesis 500. The endoprosthesis 500 may include two intramedullary stems 508a, 508b to anchor the endoprosthesis 500 in the distal end of an ulna bone 530a and radius bone 530b. An intramedullary stem 508 may be used as an anchoring device for the prostheses. The intramedullary stems may also be referred to as an osseointegration implant or osseointegrated implant.

Over time, the intramedullary stem fuses with the surrounding bone tissue, providing a strong and stable anchoring point for the rest of the endoprosthesis 500. The intramedullary stem is inserted into a hole drilled into the bone, and over time, the bone tissue grows and fuses with the surface of the implant, creating a strong and stable bond.

The intramedullary stem may be made from a biocompatible material, such as titanium or a titanium alloy. Other materials that may be used include ceramics, such as hydroxyapatite, and synthetic polymers.

The structure of the intramedullary stem can also vary depending on the application. The intramedullary stem may be screw-shaped with threads designed to be screwed into bone tissue. In some embodiments, the intramedullary stem may be cylindrical or have other shapes. The intramedullary stem 508 may have a roughened surface. Roughened surfaces have a texture or topography that promotes bone growth and attachment. The surface roughness may be achieved by various methods, such as sandblasting, acid etching, or plasma spraying.

The surface of the intramedullary stem may be porous. The surface of the implant may also be treated with coatings or other treatments to enhance osseointegration and improve the stability of the implant. The intramedullary stem may have a bioactive glass coating. The intramedullary stem may have a titanium oxide coating. The intramedullary stem may have a zinc coating.

The intramedullary stem may be coupled to a respective prosthetic bone extension 522a, 522b. The prosthetic bone extension 522 may extend from the end of a natural bone to a joint 506 of the endoprosthesis 500. The prosthetic bone extension 522 may add length to the extremity such that it matches the length of the original extremity. In some embodiments, the length of the bone extension may be chosen such that, in combination with the rest of the endoprosthesis, the hand or extremity is the same length as the original, non-amputated forearm or extremity of the surviving symmetrical limb or extremity. For example, if a right forearm is amputated, then the bone extension may be of length such that the right limb matches the length of the unamputated or intact left forearm.

The joint 506 may be a prosthetic wrist joint. The joint 506 may be a 1-degree-of-freedom pin joint that permits wrist flexion and extension. Wrist flexion involves bending the wrist downwards, towards the palm of the hand, which shortens the distance between the hand and the forearm. This movement is produced by the flexor muscles located on the front of the forearm, which contract to pull the wrist towards the palm. Wrist extension involves bending the wrist upwards, away from the palm of the hand, which increases the distance between the hand and the forearm. This movement is produced by the extensor muscles located on the back of the forearm, which contract to pull the wrist away from the palm.

In some embodiments, such as a prosthetic wrist joint, the joint may be a two degree of freedom joint that allows other types of hand movement, such as radial and ulnar deviation. Wrist radial and ulnar deviation are movements of the wrist joint that occur in the lateral plane, which is perpendicular to the palm of the hand. These movements involve a side-to-side motion of the wrist and are also known as wrist abduction and adduction, respectively.

Wrist radial deviation, or wrist abduction, involves moving the hand and wrist towards the thumb side of the forearm. This movement is produced by the muscles located on the lateral side of the forearm, which contract to pull the wrist in a lateral direction.

Wrist ulnar deviation, or wrist adduction, involves moving the hand and wrist towards the little finger side of the forearm. This movement is produced by the muscles located on the medial side of the forearm, which contract to pull the wrist in a medial direction. The angular movement of the joint in one or more degrees of freedom may be limited. The range of motion may be limited to less than 15 degrees, less than 20 degrees, less than 25 degrees, less than 30 degrees less than 35 degrees, less than 40 degrees, less than 45 degrees, or less than 50 degrees. The range of motion may exceed 15 degrees, 20 degrees, 25 degrees, 30 degrees, 35 degrees, 40 degrees, 45 degrees, or 50 degrees.

The internal muscle-driven endoprosthesis 500 may include one or more movable portions 502 that act as the moveable extremity, such as the hand. The hand portion may be made from a biocompatible metal, such as titanium and its alloys. The hand portion may be made from stainless steel. The hand portion may be made from cobalt-chromium alloys. These metals and alloys may be used for their biocompatibility, high strength-to-weight ratio, and corrosion resistance as well as because they are well-tolerated by the body and have a low risk of causing an immune response or rejection.

The hand portion may be shorter than the biological hand or palm of the hand so that it fits within the native skin envelope surrounding the biological hand.

The internal muscle-driven endoprosthesis 500 may include a sleeve that is over-molded onto the rigid segments or moveable portions, such as the movable prosthetic portions 502, the joint, and the bone extension to provide a soft surface for the overlying skin. The sleeve provides a compliant skin-internal muscle-driven endoprosthesis 500 interface that aids in reducing the risk of skin injury, the rigid segments may be over-molded with a liquid silicone rubber.

The internal muscle-driven endoprosthesis 500 may be coated with other types of biocompatible soft materials such as polyurethane, hydrogels, collagen elastomers, or other materials.

The endoprosthesis 500 may include synthetic tendons 534, 536. The synthetic tendons may extend from a natural tendon or muscles to the moving portion of the endoprosthesis limb segment. The synthetic tendons may extend from one or more of the flexor carpus radialis, flexor carpus ulnaris, palmaris longus, exor digitorum superficialis, flexor digitorum profundus, and flexor pollicis longus. The flexor carpus radialis, flexor carpus ulnaris, palmaris longus muscles originate on the humerus and cross the forearm and extend through the wrist via tendons and insert into the bones of the hand. The flexor digitorum superficialis, flexor digitorum profundus, and flexor pollicis longus, start in the forearm and the tendons attach to phalanges (finger bones). Due to where these muscles insert, they are able to help with flexing the wrists as well as flexing their respective finger/thumb they attach.

The synthetic tendons may extend and couple to one or more coupling or mounting structures 536 on the moveable hand portion 502. A tendon from each muscle or muscle group may couple to a respective one of the mounting structures 536, which may be a ring, loop, hook, or other structure. In some embodiments, multiple synthetic tendons from different muscles or muscle groups may be coupled to a single mounting structure 536.

The synthetic tendons may be made of biocompatible synthetic materials, such as polyester, polyethylene, or polyurethane, which are designed to mimic the strength and flexibility of natural tendons. The synthetic tendons may include multiple individual strands of the synthetic tendon. A first end of the synthetic tendons may be threaded into the distal ends of the muscles that contribute to hand movement in the biological hand. A second end of the synthetic tendons may be coupled or secured to a movable portion 502 that acts as the moveable extremity, such as the mounting structures 536. A first of the synthetic tendons may be coupled to the prosthetic hand 502 on a first side such as an ulnar side. A second of the synthetic tendons may be coupled to the prosthetic hand 502 on a second side such as a radial side.

In some embodiments, a first of the synthetic tendons may be coupled to the prosthetic hand 502 on a first side such as a palmar side. A second of the synthetic tendons may be coupled to the prosthetic hand 502 on a second side such as a dorsal side.

With the internal muscle-driven wrist and hand endoprosthesis 500, the hand and wrist may act as an intact hand and wrist with respect to aspects of the efferent motor command and afferent proprioceptive feedback. For example, efferent motor commands from the nervous system cause the forearm muscles to contract. The contraction of the forearm muscles pull on the synthetic tendon 520. Which causes hand portion 502 to rotate around the joint 506.

Because the hand is an internal muscle-driven endoprosthesis, the muscles are physically connected to the moving hand and there is close coupling, or alignment, among efferent motor commands representing movement intent, the actual movement, and the sensed movement via afferent proprioceptive feedback.

When the forearm muscles attached to synthetic tendon 520 contract, the hand moves, causing the synthetic tendon 510 to stretch the muscle or muscles to which it is attached to stretch.

The afferent proprioceptive feedback resulting from the stretching of the muscle or muscles to which the synthetic tendon 510 is attached is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

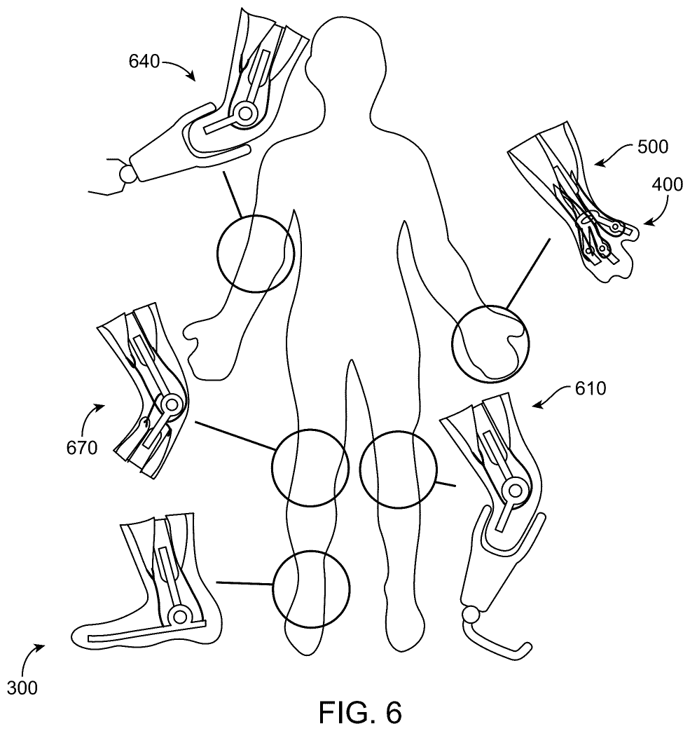

FIG. 6 shows various muscle-driven endoprosthesis. In addition to the endoprosthesis 300, 400, 500 discussed earlier, a muscle driven knee endoprosthesis 610, a muscle driven elbow endoprosthesis 640, a muscle driven knee endoprosthesis and muscle driven bridge endoprosthesis 670 are depicted.

Endoprosthesis 610 is an internal muscle-driven leg and knee endoprosthesis. The endoprosthesis 610 may include intramedullary stem to anchor the endoprosthesis 610 in the distal end of a femur bone. An intramedullary stem may be used as an anchoring device for the prostheses. The intramedullary stem may also be referred to as an osseointegration implant or osseointegrated implant.

Over time, the intramedullary stem fuses with the surrounding bone tissue, providing a strong and stable anchoring point for the rest of the endoprosthesis 610. The intramedullary stem is inserted into a hole drilled into the bone, and over time, the bone tissue grows and fuses with the surface of the implant, creating a strong and stable bond.

The intramedullary stem may be made from a biocompatible material, such as titanium or a titanium alloy. Other materials that may be used include ceramics, such as hydroxyapatite, and synthetic polymers.

The structure of the intramedullary stem can also vary depending on the application. The intramedullary stem may be screw-shaped with threads designed to be screwed into bone tissue. In some embodiments, the intramedullary stem may be cylindrical or have other shapes. The intramedullary stem may have a roughened surface. Roughened surfaces have a texture or topography that promotes bone growth and attachment. The surface roughness may be achieved by various methods, such as sandblasting, acid etching, or plasma spraying.

The surface of the intramedullary stem may be porous. The surface of the implant may also be treated with coatings or other treatments to enhance osseointegration and improve the stability of the implant. The intramedullary stem may have a bioactive glass coating. The intramedullary stem may have a titanium oxide coating. The intramedullary stem may have a zinc coating.

The intramedullary stem may be coupled to a respective prosthetic bone extension. The prosthetic bone extension may extend from the end of a natural bone, such as the femur, to a joint of the endoprosthesis 610. The prosthetic bone extension may add length to the extremity such that it matches the length of the original extremity or such that the knee joint matches the location of the original knee joint. In some embodiments, the length of the bone extension may be chosen such that, in combination with the rest of the endoprosthesis, the leg or extremity is the same length as the original, non-amputated leg or extremity of the surviving symmetrical leg or extremity. For example, if a right leg is amputated, then the bone extension may be of length such that the right leg length and knee height above ground when standing or below the hip matches the length or location of the unamputated or intact left leg and knee.

The joint may be a prosthetic knee joint. The knee joint may be a 1-degree-of-freedom pin joint that permits lower leg flexion and extension. Lower flexion involves bending the leg upwards or backwards to move the foot towards the buttocks and bringing the lower leg towards the back of the thigh. This movement is produced by the hamstring muscles, the gastrocnemius muscles, and the popliteus muscle. The hamstring muscle group is located on the back of the thigh and is composed of three muscles, the biceps femoris, the semimembranosus, and the semitendinosus. These muscles originate from the ischial tuberosity (a bony protrusion at the base of the pelvis) and insert into the tibia and fibula bones of the lower leg. The hamstring muscles are the primary muscles responsible for knee flexion and also assist in hip extension. The gastrocnemius muscle, which is located in the back of the lower leg, also plays a role in knee flexion. This muscle crosses the knee joint and assists in flexing the knee joint in addition to its primary action of plantarflexing the ankle joint. The popliteus muscle is a small muscle located on the back of the knee joint. It helps to unlock the knee joint from a fully extended position and initiate knee flexion. In some embodiments, such as a prosthetic wrist joint, the joint may be a two degree of freedom joint that allows other types of hand movement, such as radial and ulnar deviation.

Lower leg extension refers to the movement of straightening the knee joint, bringing the lower leg away from the back of the thigh. The quadriceps muscles, the tensor fasciae latae (TFL) muscle, and the iliotibial (IT) band may be involved in lower leg extension.

The quadriceps muscle group is located on the front of the thigh and is composed of four muscles: rectus femoris, vastus lateralis, vastus medialis, and vastus intermedius. These muscles originate from the pelvis and femur and insert into the patella (kneecap) and tibia bones of the lower leg via the patellar tendon. The quadriceps muscles are the primary muscles responsible for knee extension and also assist in hip flexion. The TFL muscle is a small muscle located on the side of the hip and thigh. It assists in knee extension by providing stabilization to the knee joint. The IT band is a thick band of connective tissue that runs down the side of the thigh from the hip to the knee. It assists in knee extension by providing stability to the knee joint.

The angular movement of the joint in one or more degrees of freedom may be limited. The range of motion may be limited to less than 85 degrees, less than 90 degrees, less than 95 degrees, less than 100 degrees, less than 105 degrees, less than 110 degrees, less than 115 degrees, less than 120 degrees, less than 125 degrees, less than 130 degrees, or less than 135 degrees. The range of motion exceed 85 degrees, 90 degrees, 95 degrees, 100 degrees, 105 degrees, 110 degrees, 115 degrees, 120 degrees, 125 degrees, 130 degrees, or 135 degrees.

The internal muscle-driven endoprosthesis 610 may include one or more movable portions that act as the moveable extremity, such as the lower leg. The lower leg portion may be made from a biocompatible metal, such as titanium and its alloys. The lower leg portion may be made from stainless steel. The lower leg portion may be made from cobalt-chromium alloys. These metals and alloys may be used for their biocompatibility, high strength-to-weight ratio, and corrosion resistance as well as because they are well-tolerated by the body and have a low risk of causing an immune response or rejection.

The endoprosthesis 610 may include a sleeve that is over-molded onto the rigid segments or moveable portions, such as a movable prosthetic portion, such as a lower leg portion, the joint, and the bone extension to provide a soft surface for the overlying skin. The sleeve provides a compliant skin-internal muscle-driven endoprosthesis 610 interface that aids in reducing the risk of skin injury, the rigid segments may be over-molded with a liquid silicone rubber.

The internal muscle-driven endoprosthesis 610 may be coated with other types of biocompatible soft materials such as polyurethane, hydrogels, collagen elastomers, or other materials.

The endoprosthesis may include synthetic tendons. The synthetic tendons may extend from a natural tendon or muscles to the moving portion of the endoprosthesis limb segment. The synthetic tendons may extend from one or more of the quadriceps muscles, the tensor fasciae latae (TFL) muscle, and the iliotibial (IT) band.

The synthetic tendons may extend and couple to one or more coupling or mounting structures on the moveable portion, such as the lower leg portion or the femur portion. A tendon from each muscle or muscle group may couple to a respective one of the mounting structures, which may be a ring, loop, hook, or other structure. In some embodiments, multiple synthetic tendons from different muscles or muscle groups may be coupled to a single mounting structure.

The synthetic tendons may be made of biocompatible synthetic materials, such as polyester, polyethylene, or polyurethane, which are designed to mimic the strength and flexibility of natural tendons. The synthetic tendons may include multiple individual strands of the synthetic tendon. A first end of the synthetic tendons may be threaded into the distal ends of the muscles that contribute to lower leg movement in the biological lower leg. A second end of the synthetic tendons may be coupled or secured to a movable portion that acts as the moveable extremity, such as the mounting structures. A first of the synthetic tendons may be coupled to the prosthetic lower leg portion on an anterior side. A second of the synthetic tendons may be coupled to the lower leg portion on a posterior side. A third or fourth of the synthetic tendons may be coupled to the muscles of the lower leg on a first end and to the femur on a second end.

With the internal muscle-driven knee endoprosthesis 610, the leg and knee may act as an intact leg and knee with respect to aspects of the efferent motor command and afferent proprioceptive feedback. For example, efferent motor commands from the nervous system cause the forearm muscles to contract.

Because the leg and knee have an internal muscle-driven endoprosthesis, the muscles are physically connected between the upper and lower moving leg and knee and there is close coupling, or alignment, among efferent motor commands representing movement intent, the actual movement, and the sensed movement via afferent proprioceptive feedback.

When a first of the muscles attached to a synthetic tendon contract, the leg moves, causing another of the synthetic tendons to stretch the muscle or muscles to which it is attached.

The afferent proprioceptive feedback resulting from the stretching of the muscle or muscles to which the synthetic tendon is attached is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

Endoprosthesis 640 is an internal muscle-driven arm and elbow endoprosthesis. The endoprosthesis 640 may include intramedullary stem to anchor the endoprosthesis 640 in the distal end of a humerus bone. The endoprosthesis 640 may include a second or third intramedullary stem to anchor the endoprosthesis 640 to the proximal end of the radius or ulna. An intramedullary stem may be used as an anchoring device for the prostheses. The intramedullary stem may also be referred to as an osseointegration implant or osseointegrated implant.

Over time, the intramedullary stem fuses with the surrounding bone tissue, providing a strong and stable anchoring point for the rest of the endoprosthesis 640. The intramedullary stem is inserted into a hole drilled into the bone, and over time, the bone tissue grows and fuses with the surface of the implant, creating a strong and stable bond.

The intramedullary stem may be made from a biocompatible material, such as titanium or a titanium alloy. Other materials that may be used include ceramics, such as hydroxyapatite, and synthetic polymers.

The structure of the intramedullary stem can also vary depending on the application. The intramedullary stem may be screw-shaped with threads designed to be screwed into bone tissue. In some embodiments, the intramedullary stem may be cylindrical or have other shapes. The intramedullary stem may have a roughened surface. Roughened surfaces have a texture or topography that promotes bone growth and attachment. The surface roughness may be achieved by various methods, such as sandblasting, acid etching, or plasma spraying.

The surface of the intramedullary stem may be porous. The surface of the implant may also be treated with coatings or other treatments to enhance osseointegration and improve the stability of the implant. The intramedullary stem may have a bioactive glass coating. The intramedullary stem may have a titanium oxide coating. The intramedullary stem may have a zinc coating.

The intramedullary stem may be coupled to a respective prosthetic bone extension. The prosthetic bone extension may extend from the end of a natural bone, such as the humerus, to a joint of the endoprosthesis 640. The prosthetic bone extension may add length to the extremity such that it matches the length of the original extremity or such that the elbow joint matches the location of the original elbow joint. In some embodiments, the length of the bone extension may be chosen such that, in combination with the rest of the endoprosthesis, the arm or extremity is the same length as the original, non-amputated arm or extremity of the surviving symmetrical arm or extremity. For example, if a right arm is amputated, then the bone extension may be of length such that the right arm length and elbow distance from the shoulder matches the length or location of the unamputated or intact left arm and elbow.

The joint may be a prosthetic elbow joint. The elbow joint may be a 1-degree-of-freedom pin joint that permits lower arm flexion and extension. Arm flexion refers to the movement of bending the arm at the elbow joint, bringing the hand closer to the shoulder. This movement is produced in part by the biceps brachii muscles and the brachialis muscles. The biceps brachii muscle is located on the front of the upper arm and is composed of two heads, the short head and the long head. These heads originate from the scapula and join together to insert into the radius bone of the forearm. The biceps brachii is the primary muscle responsible for arm flexion. The brachialis muscle is located on the front of the upper arm, beneath the biceps brachii muscle. It originates from the lower half of the humerus bone of the upper arm and inserts into the ulna bone of the forearm. The brachialis muscle is also involved in arm flexion, particularly when the arm is in a pronated position (palm facing downwards).

Arm extension refers to the movement of straightening the arm at the elbow joint, bringing the hand away from the shoulder. This movement is produced by the triceps brachii muscle and the anconeus muscle. The triceps brachii muscle is located on the back of the upper arm and is composed of three heads: the long head, lateral head, and medial head. These heads originate from the scapula and humerus bones of the upper arm and join together to insert into the ulna bone of the forearm. The triceps brachii is the primary muscle responsible for arm extension. The anconeus muscle is a small muscle located on the back of the elbow joint. It originates from the humerus bone and inserts into the ulna bone. The anconeus muscle assists the triceps brachii muscle in arm extension.

The angular movement of the joint in one or more degrees of freedom may be limited. The range of motion may be limited to less than 85 degrees, less than 90 degrees, less than 95 degrees, less than 100 degrees, less than 105 degrees, less than 110 degrees, less than 115 degrees, less than 120 degrees, less than 125 degrees, less than 130 degrees, or less than 135 degrees. The range of motion exceed 85 degrees, 90 degrees, 95 degrees, 100 degrees, 105 degrees, 110 degrees, 115 degrees, 120 degrees, 125 degrees, 130 degrees, or 135 degrees.

The internal muscle-driven endoprosthesis 640 may include one or more movable portions that act as the moveable extremity, such as the lower arm. The lower arm portion may be made from a biocompatible metal, such as titanium and its alloys. The lower arm portion may be made from stainless steel. The lower arm portion may be made from cobalt-chromium alloys. These metals and alloys may be used for their biocompatibility, high strength-to-weight ratio, and corrosion resistance as well as because they are well-tolerated by the body and have a low risk of causing an immune response or rejection.

The endoprosthesis 640 may include a sleeve that is over-molded onto the rigid segments or moveable portions, such as a movable prosthetic portion, such as a lower arm portion, the joint, and the bone extension to provide a soft surface for the overlying skin. The sleeve provides a compliant skin-internal muscle-driven endoprosthesis 640 interface that aids in reducing the risk of skin injury, the rigid segments may be over-molded with a liquid silicone rubber.

The internal muscle-driven endoprosthesis 640 may be coated with other types of biocompatible soft materials such as polyurethane, hydrogels, collagen elastomers, or other materials.

The endoprosthesis may include synthetic tendons. The synthetic tendons may extend from a natural tendon or muscles to the moving portion of the endoprosthesis limb segment. The synthetic tendons may extend from one or more of the biceps brachii muscles and the brachialis muscles.

The synthetic tendons may extend from a natural tendon or muscles to the humerus portion of the endoprosthesis limb segment. The synthetic tendons may extend from one or more of the lower arm muscles, such as the flexor digitorum, flexor pollicis, the brachioradialis, or other muscles of the lower arm to the humerus portion.

The synthetic tendons may extend and couple to one or more coupling or mounting structures on the moveable portion, such as the lower arm portion or the humerus portion. A tendon from each muscle or muscle group may couple to a respective one of the mounting structures, which may be a ring, loop, hook, or other structure. In some embodiments, multiple synthetic tendons from different muscles or muscle groups may be coupled to a single mounting structure.

The synthetic tendons may be made of biocompatible synthetic materials, such as polyester, polyethylene, or polyurethane, which are designed to mimic the strength and flexibility of natural tendons. The synthetic tendons may include multiple individual strands of the synthetic tendon. A first end of the synthetic tendons may be threaded into the distal ends of the muscles that contribute to lower arm movement in the biological lower arm. A second end of the synthetic tendons may be coupled or secured to a movable portion that acts as the moveable extremity, such as the mounting structures. A first of the synthetic tendons may be coupled to the prosthetic lower arm portion on an anterior side. A second of the synthetic tendons may be coupled to the lower arm portion on a posterior side. A third or fourth of the synthetic tendons may be coupled to the muscles of the lower leg on a first end and to the femur on a second end.

With the internal muscle-driven elbow endoprosthesis 640, the arm and elbow may act as an intact leg and knee with respect to aspects of the efferent motor command and afferent proprioceptive feedback. For example, efferent motor commands from the nervous system cause the forearm muscles to contract.

Because the arm and elbow have an internal muscle-driven endoprosthesis, the muscles are physically connected between the upper and lower moving arm and elbow and there is close coupling, or alignment, among efferent motor commands representing movement intent, the actual movement, and the sensed movement via afferent proprioceptive feedback.

When a first of the muscles attached to a synthetic tendon contract, the arm moves, causing another of the synthetic tendons to stretch the muscle or muscles to which it is attached.

The afferent proprioceptive feedback resulting from the stretching of the muscle or muscles to which the synthetic tendon is attached is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

Endoprosthesis 670 is an internal muscle-driven leg and knee endoprosthesis with an intact lower leg. The endoprosthesis 670 may include intramedullary stem to anchor the endoprosthesis 670 in the distal end of a femur bone. Second and third intramedullary stems may be used to anchor the endoprosthesis 670 to the tibia and fibula. An intramedullary stem may be used as an anchoring device for the prostheses. The intramedullary stem may also be referred to as an osseointegration implant or osseointegrated implant.

Over time, the intramedullary stem fuses with the surrounding bone tissue, providing a strong and stable anchoring point for the rest of the endoprosthesis 670. The intramedullary stem is inserted into a hole drilled into the bone, and over time, the bone tissue grows and fuses with the surface of the implant, creating a strong and stable bond.

The intramedullary stem may be made from a biocompatible material, such as titanium or a titanium alloy. Other materials that may be used include ceramics, such as hydroxyapatite, and synthetic polymers.

The structure of the intramedullary stem can also vary depending on the application. The intramedullary stem may be screw-shaped with threads designed to be screwed into bone tissue. In some embodiments, the intramedullary stem may be cylindrical or have other shapes. The intramedullary stem may have a roughened surface. Roughened surfaces have a texture or topography that promotes bone growth and attachment. The surface roughness may be achieved by various methods, such as sandblasting, acid etching, or plasma spraying.

The surface of the intramedullary stem may be porous. The surface of the implant may also be treated with coatings or other treatments to enhance osseointegration and improve the stability of the implant. The intramedullary stem may have a bioactive glass coating. The intramedullary stem may have a titanium oxide coating. The intramedullary stem may have a zinc coating.

The intramedullary stem may be coupled to a respective prosthetic bone extension. The prosthetic bone extension may extend from the end of a natural bone, such as the femur, to a joint of the endoprosthesis 670. Additional bone extensions may extend from the end of the tibia and fibula. The prosthetic bone extension may add length to the extremity such that it matches the length of the original extremity or such that the knee joint matches the location of the original knee joint. In some embodiments, the length of the bone extension may be chosen such that, in combination with the rest of the endoprosthesis, the leg or extremity is the same length as the original, non-amputated leg or extremity of the surviving symmetrical leg or extremity. For example, if a right leg is amputated, then the bone extension may be of length such that the right leg length and knee height above ground when standing or below the hip matches the length or location of the unamputated or intact left leg and knee.

The joint may be a prosthetic knee joint. The knee joint may be a 1-degree-of-freedom pin joint that permits lower leg flexion and extension. Lower flexion involves bending the leg upwards or backwards to move the foot towards the buttocks and bringing the lower leg towards the back of the thigh. This movement is produced by the hamstring muscles, the gastrocnemius muscles, and the popliteus muscle. The hamstring muscle group is located on the back of the thigh and is composed of three muscles, the biceps femoris, the semimembranosus, and the semitendinosus. These muscles originate from the ischial tuberosity (a bony protrusion at the base of the pelvis) and insert into the tibia and fibula bones of the lower leg. The hamstring muscles are the primary muscles responsible for knee flexion and also assist in hip extension. The gastrocnemius muscle, which is located in the back of the lower leg, also plays a role in knee flexion. This muscle crosses the knee joint and assists in flexing the knee joint in addition to its primary action of plantarflexing the ankle joint. The popliteus muscle is a small muscle located on the back of the knee joint. It helps to unlock the knee joint from a fully extended position and initiate knee flexion. In some embodiments, such as a prosthetic wrist joint, the joint may be a two degree of freedom joint that allows other types of hand movement, such as radial and ulnar deviation.

Lower leg extension refers to the movement of straightening the knee joint, bringing the lower leg away from the back of the thigh. The quadriceps muscles, the tensor fasciae latae (TFL) muscle, and the iliotibial (IT) band may be involved in lower leg extension.

The quadriceps muscle group is located on the front of the thigh and is composed of four muscles: rectus femoris, vastus lateralis, vastus medialis, and vastus intermedius. These muscles originate from the pelvis and femur and insert into the patella (kneecap) and tibia bones of the lower leg via the patellar tendon. The quadriceps muscles are the primary muscles responsible for knee extension and also assist in hip flexion. The TFL muscle is a small muscle located on the side of the hip and thigh. It assists in knee extension by providing stabilization to the knee joint. The IT band is a thick band of connective tissue that runs down the side of the thigh from the hip to the knee. It assists in knee extension by providing stability to the knee joint.

The angular movement of the joint in one or more degrees of freedom may be limited. The range of motion may be limited to less than 85 degrees, less than 90 degrees, less than 95 degrees, less than 100 degrees, less than 105 degrees, less than 110 degrees, less than 115 degrees, less than 120 degrees, less than 125 degrees, less than 130 degrees, or less than 135 degrees. The range of motion exceed 85 degrees, 90 degrees, 95 degrees, 100 degrees, 105 degrees, 110 degrees, 115 degrees, 120 degrees, 125 degrees, 130 degrees, or 135 degrees.

The internal muscle-driven endoprosthesis 670 may include one or more movable portions that act as the moveable extremity, such as the lower leg. The lower leg portion may be made from a biocompatible metal, such as titanium and its alloys. The lower leg portion may be made from stainless steel. The lower leg portion may be made from cobalt-chromium alloys. These metals and alloys may be used for their biocompatibility, high strength-to-weight ratio, and corrosion resistance as well as because they are well-tolerated by the body and have a low risk of causing an immune response or rejection.

The endoprosthesis 670 may include a sleeve that is over-molded onto the rigid segments or moveable portions, such as a movable prosthetic portion, such as a lower leg portion, the joint, and the bone extension to provide a soft surface for the overlying skin. The sleeve provides a compliant skin-internal muscle-driven endoprosthesis 670 interface that aids in reducing the risk of skin injury, the rigid segments may be over-molded with a liquid silicone rubber.

The internal muscle-driven endoprosthesis 670 may be coated with other types of biocompatible soft materials such as polyurethane, hydrogels, collagen elastomers, or other materials.

The endoprosthesis may include synthetic tendons. The synthetic tendons may extend from a natural tendon or muscles to the moving portion of the endoprosthesis limb segment. The synthetic tendons may extend from one or more of the quadriceps muscles, the tensor fasciae latae (TFL) muscle, and the iliotibial (IT) band.

Additional synthetic tendons may extend from a natural tendon or muscles to the femur portion of the endoprosthesis limb segment. The synthetic tendons may extend from one or more of the lower leg muscles, such as the gastrocnemius to the femur portion.

The synthetic tendons may extend and couple to one or more coupling or mounting structures on the moveable portion, such as the lower leg portion or the femur portion. A tendon from each muscle or muscle group may couple to a respective one of the mounting structures, which may be a ring, loop, hook, or other structure. In some embodiments, multiple synthetic tendons from different muscles or muscle groups may be coupled to a single mounting structure.

The synthetic tendons may be made of biocompatible synthetic materials, such as polyester, polyethylene, or polyurethane, which are designed to mimic the strength and flexibility of natural tendons. The synthetic tendons may include multiple individual strands of the synthetic tendon. A first end of the synthetic tendons may be threaded into the distal ends of the muscles that contribute to lower leg movement in the biological lower leg. A second end of the synthetic tendons may be coupled or secured to a movable portion that acts as the moveable extremity, such as the mounting structures. A first of the synthetic tendons may be coupled to the prosthetic lower leg portion on an anterior side. A second of the synthetic tendons may be coupled to the lower leg portion on a posterior side. A third or fourth of the synthetic tendons may be coupled to the muscles of the lower leg on a first end and to the femur on a second end.

With the internal muscle-driven knee endoprosthesis 610, the leg and knee may act as an intact leg and knee with respect to aspects of the efferent motor command and afferent proprioceptive feedback. For example, efferent motor commands from the nervous system cause the forearm muscles to contract.

Because the leg and knee have an internal muscle-driven endoprosthesis, the muscles are physically connected between the upper and lower moving leg and knee and there is close coupling, or alignment, among efferent motor commands representing movement intent, the actual movement, and the sensed movement via afferent proprioceptive feedback.

When a first of the muscles attached to a synthetic tendon contract, the leg moves, causing another of the synthetic tendons to stretch the muscle or muscles to which it is attached.

The afferent proprioceptive feedback resulting from the stretching of the muscle or muscles to which the synthetic tendon is attached is provided by specialized sensory receptors known as proprioceptors, which are located in muscles, tendons, and joints. These receptors detect mechanical stimuli such as tension, stretch, and pressure, and generate electrical signals that travel through afferent nerve fibers to the spinal cord and brainstem, where they are processed and integrated to produce a perceptual representation of the body's position and movement.

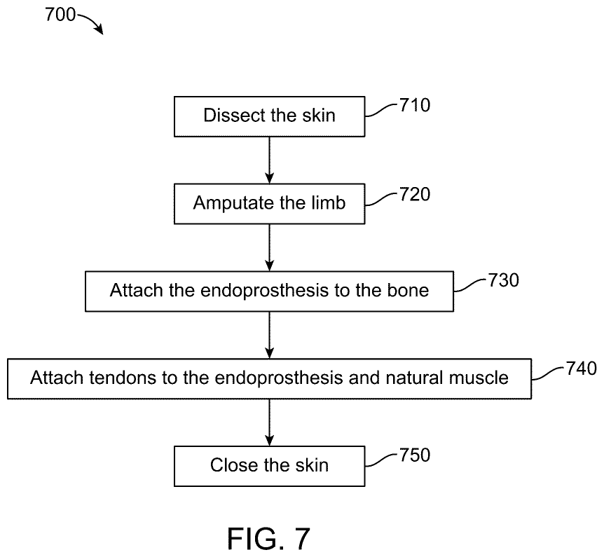

FIG. 7 shows a method 700 of using a various muscle-driven endoprosthesis, such as those disclosed herein. The method may start at block 710 wherein the skin is dissected. The method may include amputating the existing bone and/or limb at block 720. At block 730 a muscle-driven endoprosthesis, such as those shown and described herein, may be attached to the bone. At block 740 synthetic tendons are attached to the natural ends of the muscles or existing tendons and to the endoprosthesis. At block 750 the skin is closed around the muscle-driven endoprosthesis.

At block 710 the skin is dissected. A circumferential incision may be made at a location between the skin and tissue to be retained and used to cover the endoprosthesis and the tissue to be amputated and removed. A longitudinal linear incision may be made along the length of the limb to open the skin that is retained for covering the prosthesis to allow surgical access to the tissues, such as the retained bone and muscles. In some embodiments such as those which retain the limb, such as elbow and knee endoprosthesis with retained upper and lower limb portions, the incision may include a circumferential with longitudinal incisions up and down the limb from the incision.

At block 720 the limb is amputated. The muscles and bones that are not retainer are amputated. The amputated bone may include proximal or distal ends of bones. The end of the bone to be amputated may depend on the type of amputation and endoprosthesis used. For example, a knee or elbow endoprosthesis for use with existing upper and lower legs or arms may include the amputation of the lower femur or humerus and the upper tibia, fibula, radius, and/or ulna while retaining at least a portion of each bone Amputation may also include preparing the bone for an intramedullary stem, such as by forming a hole or in which the intramedullary stem may be inserted.

At block 730 the muscle-driven endoprosthesis, such as those shown and described herein, may be attached to the bone. An intramedullary stem may be used as an anchoring device for the endoprosthesis. The intramedullary stem is inserted into the bone to couple the endoprosthesis to the patient's anatomy. Over time, the intramedullary stem fuses with the surrounding bone tissue, providing a strong and stable anchoring point for the rest of the endoprosthesis.

At block 740 synthetic tendons are attached to the natural ends of the muscles or existing tendons and to the endoprosthesis. The synthetic tendons may extend and couple from a natural muscle or tendon to one or more coupling or mounting structures on the endoprosthesis. A tendon from each muscle or muscle group may be coupled to a respective one of the mounting structures. In some embodiments, multiple synthetic tendons from different muscles or muscle groups may be coupled to a single mounting structure.

A synthetic tendon or portion thereof may be inserted individual through an end of the retained muscle or tendon then sewn through the muscle or tendon, exiting the muscle about 1 cm from the insertion location. Each synthetic tendon or portion thereof may be sewn into the muscle within about 1 cm or within about 1.5 cm of each other. The individual synthetic suture portions may be coupled together along their length, such as by tying, weaving, or bundling. The synthetic tendon may be coupled to the endoprosthesis, as described herein.

At block 750 the skin is closed around the endoprosthesis. In some embodiments, a skin graft may be used to close the skin around the endoprosthesis. In some embodiments, the skin graft may be a natural skin graft of the patient or from a donor. In some embodiments, the skin graft may be a heterograft. In some embodiments, the skin graft may be a synthetic skin substitute, such as manufactured skin equivalent.

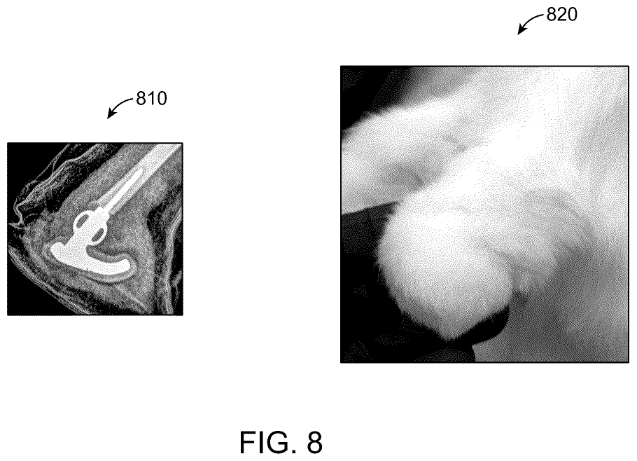

FIG. 8 shows an experimental muscle-driven endoprosthesis 810. Several experimental animal studies have been conducted that demonstrating the feasibility of the muscle driven endoprostheses. A custom foot-ankle endoprosthesis prototype using computer-aided design (CAD) software. The dimensions of the foot segment were constrained based on the pre-determined size of skin flaps that preserved blood perfusion throughout the flap. The prototype was designed and printed as one piece with the ankle segment unjointed (i.e., unable to rotate). The intramedullary stem and core were made using 316L stainless steel. The core was over-molded with biocompatible silicone (BIO M340, Elkem Silicones). The amputation and prosthesis implantation were performed in the same surgery. In each of four rabbits, the skin surrounding the proximal biological foot and ankle was dissected and reflected proximally. The tibia was transected about 2 cm proximal to the ankle joint, and distal tissues were removed. The stem was anchored in the tibia intramedullary canal with bone cement. For added stability, the tibialis cranialis insertion tendon and Achilles tendon were sutured to eyelets on the cranial and caudal aspects, respectively, of the endoprosthesis. The skin flap was replaced over the endoprosthesis and sutured closed. The rabbits were studied for 60 days post-surgery at which time tissues were harvested.

Three rabbits survived to the pre-defined 60-day study endpoint with no complications. The sutured edges of the skin fully healed and closed. Fur regrew over all the skin covering the prosthesis by about 30 days post-surgery. At 60 days post-surgery 820, the skin appeared healthy. Upon dissection, we observed that a fibrous capsule had formed over the prosthesis.

The proof-of-concept study demonstrated the viability of the skin flap for covering an endoprosthesis with a foot segment, supporting the feasibility of implanting a jointed foot-ankle endoprosthesis with muscle attachment. An endoprosthesis is similar to traditional orthopedic joint replacements except that the latter are typically placed between intact biological limb segments. Conversely, the endoprosthesis extends from the distal end of a limb segment. The observed fibrous capsule had a benign effect on the unjointed endoprosthesis but may restrict the range of motion of a jointed endoprosthesis. Early post-operative mobilization of a jointed endoprosthesis may be needed to limit or prevent capsule formation.

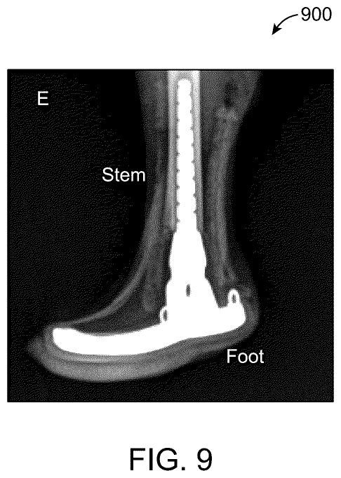

FIG. 9 an experimental jointed muscle-driven endoprosthesis 900. The custom foot-ankle endoprosthesis prototypes included 3D-printed 316 stainless-steel foot and shank segments. The segments were joined by a polyethylene hinge pin and over molded in medical-grade silicone (BIO M360, Elkem Silicones). Two 18-week-old female New Zealand White rabbits underwent surgical amputation of the left hindlimb under general anesthesia. Briefly, the skin surrounding the foot and ankle was retracted proximally. The musculoskeletal tissues were amputated approximately 2 cm proximal to the distal end of the tibia. After amputation, the prosthesis was anchored in the intramedullary canal of the tibia and immobilized using bone cement. The tibialis cranialis and triceps surae muscles were attached to eyelets on the foot segment using polyester-based artificial tendons in Rabbit 1 and the native tendons in Rabbit 2. The skin was replaced and sutured over the prosthesis.

Once bandages were removed (3 weeks post-surgery), we recorded ground contact pressures (HR Strideway System, Tekscan, Inc.) and kinematics during hopping gait. We used off-the-shelf software (Strideway Research 7.80, Tekscan, Inc.) to process the pressure data.

Stance time on the operated limb initially decreased from pre-surgery (−1 week) to 3 weeks post-surgery, but then improved from 3 to 7 weeks post-surgery in both rabbits. The normalized peak vertical force gradually decreased in the operated limb from pre-surgery to 7 weeks post-surgery in Rabbit 1 (artificial tendons). Conversely, in Rabbit 2 (native tendons), force sharply declined initially but improved from 3 to 7 weeks post-surgery. In both rabbits, normalized peak force in the non-operated limb initially increased but returned towards baseline by 7 weeks post-surgery. The lower force at post-surgery week 3 in Rabbit 2 may have been due to high stiffness and low range of motion in the ankle joint during the first 4-6 weeks post-surgery.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Although illustrated as separate elements, the method steps described and/or illustrated herein may represent portions of a single method.

A person of ordinary skill in the art will recognize that any process, system, or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising”.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A muscle-driven endoprosthesis system comprising:
a bone extension;
an intramedullary stem extending form a first end of the bone extension;
a joint at a second end of the bone extension;
a moveable portion coupled to the hinge; and
synthetic tendons coupled to the moveable portion,
wherein the bone extension, intramedullary stem, hinge, and movable portion are sized and shaped to fit within a skin of a patient.

2. The muscle-driven endoprosthesis system of claim 1, wherein:
a diameter of the intramedullary stem is less than a diameter of the bone extension.

3. The muscle-driven endoprosthesis system of claim 1, wherein:
the moveable portion is a biocompatible material.

4. The muscle-driven endoprosthesis system of claim 1, wherein:
the bone extension is a biocompatible material.

5. The muscle-driven endoprosthesis system of claim 1, wherein:
the joint is a 1-degree-of-freedom hinge joint.

6. The muscle-driven endoprosthesis system of claim 5, wherein:
an angular movement of the joint allows movement between 0 and 135 degrees.

7. The muscle-driven endoprosthesis system of claim 1, wherein:
the joint is a multiple-degree-of-freedom joint.

8. The muscle-driven endoprosthesis system of claim 1, wherein:
the synthetic tendons include a first synthetic tendon coupled to the movement portion on a first side of the joint and a second synthetic tendon coupled to the moveable portion on a second side of the joint.

9. The muscle-driven endoprosthesis system of claim 1, wherein:
the synthetic tendons include a multiple individual strands of biocompatible material.

10. The muscle-driven endoprosthesis system of claim 9, wherein:
the multiple strands of biocompatible material are coupled together along their length.

11. The muscle-driven endoprosthesis system of claim 1, further comprising:
a plurality of tendon attachment structures on the moveable portion.

12. The muscle-driven endoprosthesis system of claim 11, wherein:
the plurality of tendon attachment structures define an aperture to receive a first of the plurality of synthetic tendons.

13. The muscle-driven endoprosthesis system of claim 1, further comprising:
a sleeve over-molded onto the moveable portion.

14. The muscle-driven endoprosthesis system of claim 13, wherein:
the sleeve is a compliant biocompatible material.

15. The muscle-driven endoprosthesis system of claim 13, wherein:
the sleeve is a silicone rubber.

16. The muscle-driven endoprosthesis system of claim 13, wherein:
the sleeve is between 1 mm and 6 mm thick.

17. The muscle-driven endoprosthesis system of claim 1, wherein:
the moveable portion includes a first portion that extends from the joint in an anterior direction and a second portion that extends from the joint in a posterior direction.

18. The muscle-driven endoprosthesis system of claim 17, wherein:
the first portion has a first length and the second portion has a second length, wherein the first length is greater than the second length.

19. The muscle-driven endoprosthesis system of claim 17, wherein:
the first length of the first portion extends in a direction perpendicular to the bone extension.

20. The muscle-driven endoprosthesis system of claim 1, further comprising:
a second joint, the second joint being at a distal end of the moveable portion.
